(19) United States
(12) Patent Application Publication
(10) Pub. No.: US 2012/0121722 A1
(43) Pub. Date: May 17, 2012

(54) ATAZANAVIR FORMULATIONS

(76) Inventors: Anup Avijit Choudhury, Khurda (IN); Ashish Jaiswal, Indore (IN); Khalid Rafi, New Delhi (IN); Rajeev Raghuvanshi, Gurgaon (IN)

(21) Appl. No.: 13/140,154

(22) PCT Filed: Dec. 17, 2009
(86) PCT No.: PCT/IB09/55830

§ 371 Date: Feb. 6, 2012

(30) Foreign Application Priority Data
Dec. 18, 2008 (IN) 2894/DEL/2008

(51) Int. Cl. A61K 33/00; B29B 9/10

(52) U.S. Cl. 424/600; 264/6

(57) ABSTRACT

The present invention relates to formulations comprising atazanavir and a silicate, and the process of preparation thereof, wherein the silicate is present in the extragranular portion.

### FIELD OF THE INVENTION

The present invention relates to formulations comprising atazanavir and a silicate, and the process of preparation thereof, wherein the silicate is present in the extragranular portion.

### BACKGROUND OF THE INVENTION

Atazanavir is an azapeptide protease inhibitor which selectively inhibits the virus-specific processing of viral gag-pol polyproteins in HIV-1 infected cells. It is commercialized in the form of its sulfate salt, and is available under the trade name REYATAZ® in 100 mg, 150 mg, 200 mg and 300 mg capsules from Bristol-Myers Squibb Company, USA. U.S. Pat. No. 5,849,911 discloses atazanavir and its use in AIDS therapy. U.S. Pat. No. 6,087,383 specifically describes atazanavir sulfate, and the process of preparation thereof.

Atazanavir is a substrate of the cytochrome P450 3A4 isoenzyme, leading to unfavourable pharmacokinetics of the drug upon oral administration. Consequently, co-administration of atazanavir with an agent that inhibits metabolism by cytochrome P450 monooxygenase will improve the pharmacokinetics (i.e., increase the half-life, increase the time to peak plasma concentration, increase the blood levels) of atazanavir leading to beneficial therapeutic effects. Such pharmacokinetic property-improving action of ritonavir when subjected to co-administration with drugs susceptible to metabolism by cytochrome P450 enzymes has been illustrated vividly in WO 97/01349. Therefore, ritonavir is used as a booster in atazanavir pharmacokinetics as it has been shown to significantly increase the pharmacokinetic parameters of atazanavir. It is also recommended that 300 mg atazanavir and 100 mg ritonavir should be taken once daily, with food, for treatment-experienced patients.

There have been several publications describing pharmaceutical formulations containing protease inhibitors. WO 95/09614 discloses a solid pharmaceutical formulation of a HIV protease inhibiting compounds which comprises a mixture of protease inhibitor, adsorbents, an organic solvent and a combination of pharmaceutically acceptable acids. WO 01/34118 and WO 01/34119 describe solid dispersion formulations comprising HIV protease inhibitor(s), and process for their preparation. Further, WO 2005/039551 discloses another solid dispersion formulation which exhibits improved bioavailability.

It is always desirable that a solid dosage form undergo rapid disintegration such that it releases the active ingredient into the body quickly, which in turn results in a rapid onset of therapeutic action. A tablet dosage form should be comprised of excipients that are compatible with the active ingredient and should have appropriate disintegration characteristics to ensure rapid release of the drug from the formulation and consequently appropriate dissolution characteristics.

While formulating atazanavir tablets, the present inventors found that the use of conventional disintegrants did not satisfy the disintegration characteristics. In fact, it was observed that atazanavir tablets when subjected to in vitro studies, instead of disintegrating, formed a lump and exhibited extremely poor dissolution. EP 1 800 681 describes the use of silicates in solid pharmaceutical composition comprising ritonavir and atazanavir. Silicate compounds are used in these formulations in the intragranular portion, as a compound which enables gastrointestinal fluid to penetrate that composition. However, the present inventors found that use of intragranular silicate in atazanavir formulations also did not yield desirable results with respect to disintegration and dissolution. However, when silicates were used in the extragranular portion, the resulting formulations exhibited effective disintegration and consequently appropriate release characteristics. It was surprisingly found that the presence of extragranular silicates lead to appropriate disintegration and consequently superior dissolution properties.

### SUMMARY OF THE INVENTION

In one general aspect the present invention includes a pharmaceutical composition which includes an intragranular portion comprising atazanavir; an extragranular portion comprising a silicate; and one or more of pharmaceutically acceptable excipient(s).

The invention may include one or more of the following features; for example, the silicate may be calcium silicate. The silicate may be present in an amount from about 1% (w/w) to about 20% (w/w) by weight of the formulation.

The composition may release at least about 50% of atazanavir in 5 minutes when subjected to in vitro dissolution in a USP type II apparatus, at 50 rpm, at a temperature of 37° C±0.5° C. in 1000 mL of 0.025 N hydrochloric acid.

The pharmaceutically acceptable excipient(s) may include one or more of diluent(s), binder(s), disintegrant(s), lubricant(s), and glidant(s). Further, the pharmaceutical composition may also include ritonavir.

In another embodiment, the present invention includes a process of preparation of a pharmaceutical composition of atazanavir. The process includes blending atazanavir with one or more of pharmaceutically acceptable excipient(s), in a suitable mixer; granulating the blend of step (a) with a granulating fluid; drying or sizing the granules of step (b); mixing the sized granules of step (c) with a silicate; and mixing the blend of step (d) with one or more of pharmaceutically acceptable excipient(s). The pharmaceutical composition prepared according to this invention may be compressed into a tablet or filled into a capsule.

### DETAILED DESCRIPTION OF THE INVENTION

The term “pharmaceutical formulation”, as described herein, encompasses without limitation intimate or non-intimate blends, tablets, minitablets or capsules.

Atazanavir, includes pharmaceutically acceptable salts, solvates, enantiomers, diastereomers, and polymorphs thereof. In one embodiment, the preferred salt form of atazanavir may be atazanavir sulphate.

Atazanavir and/or ritonavir present in the pharmaceutical formulation is in amounts suitable to elicit a particular clinical response being sought by the person skilled in the art. The amount may be a “therapeutically effective amount”, i.e., atazanavir or ritonavir in amounts that result in the alleviation of the symptoms of the disease or condition being treated by the drug. The amount may be a “prophylactically effective amount”, i.e., atazanavir or ritonavir in amounts that result in prophylaxis of the symptoms of the disease or condition being prevented by the drug. The amount also refers to an amount that would provide enhanced therapeutic activity of another drug that is co-administered with it, in a way that if the later drug was administered alone, would not have achieved the desired response, (e.g., unsatisfactory pharmacokinetic values for the drug and/or an unsatisfactory drug circulation level resulting in little or no efficacy).

The amount of atazanavir in the pharmaceutical formulation may be in the range from about 100 mg to about 1000 mg and the amount of ritonavir may be in the range from about 10 mg to about 500 mg. In one embodiment, the amount of atazanavir may be 300 mg.

The term “silicate” includes silicic acid; alkali metal silicates, such as sodium silicate and potassium silicate; alkaline earth metal silicates, such as magnesium silicate and calcium silicate; silicic acid-aluminium complex compounds, such as silicic acid-alumina; aluminium-magnesium complex compounds, such as magnesium aluminosilicate and magnesium aluminometasilicate; or mixtures thereof. In one embodiment, the silicate may be calcium silicate, such as that sold under the trade name Hubersorb®. The amount of the silicate present may range from about 1% (w/w) to about 20% (w/w), preferably from about 1% (w/w) to about 10% (w/w) by weight of the pharmaceutical formulation.

The term “pharmaceutically acceptable excipients”, includes conventional pharmaceutical additives known in the art, such as diluent(s), binder(s), disintegrant(s), superdisintegrant(s), lubricants(s), granulating solvent(s), glidant(s), or combinations thereof.

Diluents may be selected depending upon the compatibility with the active ingredient, including saccharides, such as lactose, dextrose, sucrose, fructose, maltose;

sugars such as mannitol, erythritol, sorbitol, xylitol and lactitol; cellulose derivatives, such as powdered cellulose, microcrystalline cellulose; starch and pregelatinized starch; dicalcium phosphate, tribasic calcium phosphate, calcium sulphate, calcium carbonate, kaolin, and the like. In one embodiment, lactose may be the diluent. Lactose may be lactose monohydrate, anhydrous lactose or spray-dried lactose.

Binders include, starch derivatives, such as corn starch and pregelatinized starch; cellulose ethers, such as carboxymethyl cellulose, methylcellulose, hydroxypropyl cellulose, hydroxypropyl methylcellulose; carboxy vinyl polymers, such as carbomers; acrylates as Eudragit®; polyvinylpyrrolidone, polyvinylpyrrolidone/vinyl acetate copolymer; xanthan gum, guar gum and other such materials routinely used in the art of pharmaceutical manufacturing.

Disintegrants and superdisintegrants include alginic acid, carboxymethylcellulose calcium, carboxymethylcellulose sodium, colloidal silicon dioxide, croscarmellose sodium, polyvinylpyrrolidone, crosslinked polyvinylpyrrolidone, guar gum, magnesium aluminium silicate, sodium starch glycolate, corn starch, potato starch, pregelatinized starch, low-substituted hydroxypropylcellulose, methylcellulose, microcrystalline cellulose, polacrilin potassium, powdered cellulose, pregelatinized starch, sodium alginate, and methacrylic acid divinylbenzene copolymer salts.

Lubricants include magnesium stearate, calcium stearate, zinc stearate, sodium stearyl fumarate, powdered stearic acid, magnesium oleate, calcium palmitate, potassium laureate, sodium suberate, vegetable oil, mineral oil, and the like. Glidants include talc, colloidal silicon dioxide, corn starch, and the like.

Suitable granulating solvents include, water, ethanol, methanol, isopropyl alcohol, methylene chloride, acetone, and the like.

The pharmaceutical formulation, as referred to herein, may further be co-processed with another composition comprising of ritonavir. The composition of ritonavir may be in the form of a solid dispersion, granules, an intimate or a non-intimate blend with pharmaceutically acceptable excipients, minitablets, microparticles, beads or pellets.

The pharmaceutical formulation, as described herein, along with a composition comprising ritonavir may be further processed to a unit dosage form. The term “unit dosage form”, includes standard pharmaceutical solid dosage forms and may be in the form of coated or uncoated tablets, multilayer tablets, capsules, pills, and the like. The pharmaceutical formulation may be compressed to a mono-, bi- or multi-layer tablet or may be filled into capsules of suitable size, using appropriate tooling.

The pharmaceutical formulation, as described herein, may be processed using conventional techniques known in the art, viz. dry and wet granulation.

In one embodiment, the pharmaceutical formulation may include
(a) atazanavir in the intragranular portion;
(b) a silicate in the extragranular portion; and
(c) pharmaceutically acceptable excipients comprising of diluent(s), binder(s), disitengrant(s), lubricant(s) and glidant(s).

In another embodiment, the pharmaceutical formulation may include
(a) atazanavir in the intragranular portion;
(b) a silicate in the extragranular portion; and
(c) pharmaceutically acceptable excipients comprising of diluent(s), binder(s), disitengrant(s), lubricant(s) and glidant(s),
   wherein the formulation further includes ritonavir.

The pharmaceutical formulation may be prepared by:
(a) blending atazanavir with pharmaceutically acceptable excipient(s) including diluent(s), binder(s), and disintegrant(s) in a suitable mixer;
(b) granulating the blend of step (a) with a granulating fluid;
(c) drying and sizing the granules of step (b)
(d) mixing the sized granules of step (c) with a silicate; and
(e) mixing the blend of step (d) pharmaceutically acceptable excipient(s) including one or more diluent(s), binder(s), disintegrant(s), lubricant(s), and/or glidant(s).

In the above embodiments, the pharmaceutical formulation obtained may be filled into capsules or compressed into tablets. The tablets prepared may be further coated using conventional coating techniques known in the art.

From the above, it is apparent that various modifications and combinations of the formulations detailed in the text may be made without departing from the spirit and scope of the invention. The invention, as described herein, may be illustrated by the following examples but is not to be construed to be limiting by them.

### Examples: 1 -6

| | | Quantity in mg/tablet | | | | | |
|---|---|---|---|---|---|---|---|
| S/N | Ingredients | Example 1 | Example 2 | Example 3 | Example 4 | Example 5 | Example 6 |

Intragranular Portion:

| | | | | | | | |
|---|---|---|---|---|---|---|---|
| 1. | Atazanavir Sulphate | 341.70 | 341.70 | 341.70 | 341.70 | 341.70 | 341.70 |
| | (equivalent to 300 mg | | | | | | |
| | Atazanavir) | | | | | | |
| 2. | Lactose | 169.52 | 101.55 | 169.70 | 179.70 | 179.70 | 179.70 |
| 3. | Crosslinked | 27.50 | 13.50 | 27.50 | 37.50 | 37.50 | 37.50 |
| | Polyvinylpyrrolidone | | | | | | |
| 4. | Calcium Silicate | — | 30.00 | — | — | — | — |
| 5. | Purified Water | q.s. | q.s. | q.s. | q.s. | q.s. | q.s. |

Extragranular Portion

| | | | | | | | |
|---|---|---|---|---|---|---|---|
| 6. | Calcium Silicate | 45.00 | — | 30.00 | 15.00 | — | — |
| 7 | Crosslinked | — | 13.50 | — | — | — | 37.50 |
| | Polyvinylpyrrolidone | | | | | | |
| 8. | Polyvinylpyrrolidone | 42.50 | — | 27.50 | 17.50 | 37.50 | — |
| 9. | Microcrystalline | — | — | — | 149.22 | — | — |
| | Cellulose | | | | | | |
| 10. | Lake of Quinolene | 0.63 | — | 0.60 | 0.73 | 0.60 | 0.60 |
| | Yellow | | | | | | |
| 11. | Magnesium Stearate | 3.15 | 4.75 | 3.00 | 3.65 | 3.00 | 3.00 |

| | | | | | | |
|---|---|---|---|---|---|---|
| Total | 630.00 | 505.00 | 600.00 | 745.00 | 600.00 | 600.00 |

### Procedure:

### Example 1

Atazanavir was blended with lactose, crosslinked polyvinylpyrrolidone and granulated using an aqueous solution in rapid mixer granulator. The granules obtained were dried and sized. The sized granules were blended with calcium silicate, polyvinylpyrrolidone and Lake of Quinolene Yellow. The blend was then further blended with magnesium stearate and compressed into tablets.

### Example 2

Atazanavir was blended with lactose, crosslinked polyvinylpyrrolidone, calcium silicate and granulated using an aqueous solution in rapid mixer granulator. The granules obtained were dried and sized. The sized granules were blended with crosslinked polyvinylpyrrolidone. The blend was then further blended with magnesium stearate and compressed into tablets.

### Example 3

Atazanavir was blended with lactose, crosslinked polyvinylpyrrolidone and granulated using an aqueous solution in rapid mixer granulator. The granules obtained were dried and sized. The sized granules were blended with calcium silicate, polyvinylpyrrolidone and Lake of Quinolene Yellow. The blend was then further blended with magnesium stearate and compressed into tablets.

### Example 4

Atazanavir was blended with lactose, crosslinked polyvinylpyrrolidone and granulated using an aqueous solution in rapid mixer granulator. The granules obtained were dried and sized. The sized granules obtained were blended with calcium silicate, polyvinylpyrrolidone, microcrystalline cellulose and Lake of Quinolene Yellow. The blend was then further blended with magnesium stearate and compressed into tablets.

### Example 5

Atazanavir was blended with lactose, crosslinked polyvinylpyrrolidone and granulated using an aqueous solution in rapid mixer granulator. The granules obtained were dried and sized. The sized granules obtained were blended with polyvinylpyrrolidone and Lake of Quinolene Yellow. The blend was then further blended with magnesium stearate and compressed into tablets.

### Example 6

Atazanavir was blended with lactose, crosslinked polyvinylpyrrolidone and granulated using an aqueous solution in rapid mixer granulator. The granules obtained were dried and sized. The sized granules obtained were blended with crosslinked polyvinylpyrrolidone and Lake of Quinolene Yellow. The blend was then further blended with magnesium stearate and compressed into tablets.

Tablets in Examples 1-6 and REYATAZ® Capsules (from Bristol Myers Squibb, Batch Number 7C3015A) were subjected to in vitro dissolution studies in a USP type II apparatus, at 50 rpm, at a temperature of 37° C±0.5° C. in 1000 mL of 0.025N hydrochloric acid medium. Aliquot of the sample was withdrawn at predetermined time intervals and replaced with an equal amount of fresh media. The samples were processed and analysed suitably. Dissolution profiles of these tablets are provided in Table 1.

TABLE 1
In vitro release pattern of atazanavir from REYATAZ ® capsules (from Bristol Myers
Squibb, Batch Number 7C3015A) and tablets prepared as per compositions in Examples
1-6 in USP II apparatus in 1000 mL of 0.025N hydrochloric acid medium at 50 rpm at
a temperature of 37° C. ± 0.5° C.

| Time | Percent of Atazanavir Released from Compositions | | | | | | |
|---|---|---|---|---|---|---|---|
| (min) | REYATAZ | Example 1 | Example 2 | Example 3 | Example 4 | Example 5 | Example 6 |
| 5 | 37 | 75 | 26 | 52 | 48 | 25 | 20 |
| 10 | 91 | 93 | 59 | 85 | 82 | 47 | 36 |
| 20 | 96 | 94 | 76 | 90 | 89 | 68 | 59 |
| 30 | 98 | 99 | 86 | 96 | 96 | 81 | 76 |
| 45 | 100 | 99 | 93 | 99 | 99 | 89 | 84 |
| 60 | 100 | 99 | 93 | 99 | 100 | 99 | 99 |

The atazanavir tablets in Examples 1, 3 and 4 were prepared as per the present invention and are comprised of calcium silicate in the extragranular portion. All the three formulations exhibited at least about 50% of drug release in 5 minutes. In contrast, the tablet in Example 2, which comprised of calcium silicate in the intragranular portion or tablets in Examples 5 and 6, which did not have calcium silicate exhibited poor drug release in 5 minutes. Similarly, REYATAZ® capsules exhibited less than 50% of drug release in 5 minutes.

We claim:

1. A pharmaceutical composition comprising:
(a) an intragranular portion comprising atazanavir;
(b) an extragranular portion comprising a silicate; and
(c) one or more of pharmaceutically acceptable excipient(s).

2. The pharmaceutical composition according to claim 1, wherein the silicate comprises calcium silicate.

3. The pharmaceutical composition according to claim 1, wherein the silicate comprises an amount from about 1% (w/w) to about 20% (w/w) by weight of the formulation.

4. The pharmaceutical composition according to claim 1, wherein the composition releases at least about 50% of atazanavir in 5 minutes when subjected to in vitro dissolution in a USP type II apparatus, at 50 rpm, at a temperature of 37° C±0.5° C. in 1000 mL of 0.025 N hydrochloric acid.

5. The pharmaceutical composition according to claim 1, wherein the pharmaceutically acceptable excipient(s) comprises one or more of diluent(s), binder(s), disintegrant(s), lubricant(s), and glidant(s).

6. The pharmaceutical composition according to claim 1, further comprising ritonavir.

7. A process of preparation of a pharmaceutical composition the process comprising:
(a) blending atazanavir with one or more of pharmaceutically acceptable excipient(s), in a suitable mixer;
(b) granulating the blend of step (a) with a granulating fluid;
(c) drying or sizing the granules of step (b);
(d) mixing the sized granules of step (c) with a silicate; and
(e) mixing the blend of step (d) with one or more of pharmaceutically acceptable excipient(s).

8. The process according to claim 7, wherein the process further comprises compressing the formulation into a tablet.

9. The process according to claim 7, wherein the process further comprises filling the formulation into a capsule.
